(19) United States
(12) Patent Application Publication
(10) Pub. No.: US 2024/0316001 A1
(43) Pub. Date: Sep. 26, 2024

(54) USE OF PHARMACEUTICAL COMPOSITION COMPRISING CHLOROGENIC ACID IN PREPARATION OF DRUG FOR TREATING EARLY ALZHEIMER'S DISEASE

(71) Applicant: SICHUAN JIUZHANG BIOLOGICAL SCIENCE AND TECHNOLOGY CO., LTD., Chengdu, Sichuan (CN)

(72) Inventors: Jie ZHANG, Chengdu, Sichuan (CN); Wenbin LI, Chengdu, Sichuan (CN); Wang HUANG, Chengdu, Sichuan (CN); Fei ZHANG, Chengdu, Sichuan (CN); Ya ZHANG, Chengdu, Sichuan (CN); Min XU, Chengdu, Sichuan (CN); Liang ZHANG, Chengdu, Sichuan (CN); Yujia HUANG, Chengdu, Sichuan (CN)

(21) Appl. No.: 18/577,127

(22) PCT Filed: Jul. 6, 2022
(86) PCT No.: PCT/CN2022/104223

§ 371 Date: Jan. 5, 2024

(30) Foreign Application Priority Data
Jul. 7, 2021 (CN) 202110767242.3

(51) Int. Cl. A61K 31/216; A61K 45/06; A61P 25/28

(52) U.S. Cl. CPC A61K 31/216; A61K 45/06; A61P 25/28

(57) ABSTRACT

A pharmaceutical composition containing chlorogenic acid can be used in the preparation of a drug for treating the early Alzheimer's disease. In the pharmaceutical composition chlorogenic acid is used as a main active ingredient, and 3-coumaroylquinic acid is used as an auxiliary ingredient. The latter can effectively improve the therapeutic effect of chlorogenic acid for treating the early Alzheimer's disease.

### FIELD OF THE INVENTION

The present invention belongs to the field of biomedicine, and especially relates to the use of a pharmaceutical composition comprising chlorogenic acid in the manufacturer of a medicament for treating the early Alzheimer's disease (AD).

### BACKGROUND OF THE INVENTION

Alzheimer's disease is a progressive neurodegenerative disease that is age-related, and characterized by symptoms such as agnosia, aphasia, apraxia, aphasis spatial disorientation, memory disorders, and cognitive dysfunction. The gross pathological manifestations of the patient's brain include reduced weight and volume, wider and deeper sulci, gyral atrophy, and temporal lobe atrophy, especially hippocampal atrophy. Its main histopathological feature is aging plaques formed by argyrophilic axonal processes wrapping around β-amyloid proteins, neurofibrillary tangles formed by highly phosphorylated microtubule tau protein in neurons, gliosis in the brain, and neuronal loss. The epidemiological survey results show that the total number of people affected worldwide is expected to increase to 100 million by 2050.

At present, many speculations have been formed about the pathological characteristics of AD, such as oxidative stress theory, amyloid hypothesis, inflammatory mechanism, insulin-related abnormal glucose metabolism hypothesis, free radical hypothesis, etc. The large amount of high-density Aβ and its neurotoxic effects in the brain of patients with AD are one of the important causes of AD, which has become an indisputable fact. The pathological changes of neuronal tau protein coexist with Aβ deposition and can produce various inflammatory reactions. Meanwhile, there is ample evidence to suggest that oxidative stress, chronic neuroinflammatory injury, neuronal apoptosis and autophagy form a complex network with Aβ deposition, abnormal phosphorylation of tau protein, and the resulting neurotoxicity.

Currently, medicaments that have been marketed for the treatment of AD mainly focus on assisting in improvement of disease symptoms, but cannot fundamentally prevent the deterioration of the conditions. In clinical practice, the treatment of AD often involves the use of cholinesterase inhibitors such as galantamine, antioxidant drugs such as monoamine oxidase inhibitors, and cerebral metabolic activators (CMA) such as oxiracetam, but the results are not satisfactory. In fact, in the past few decades, the development of new drugs in this field has almost completely failed. Several drugs of pharmaceutical giants such as Pfizer and AstraZeneca have suffered failures in phase 3 clinical trials. Natural plants contain abundant drug lead-compounds as well as compounds with unique mechanisms of action and novel structures. Numerous studies have found that certain components in natural medicines have effects such as promoting Aβ elimination, reducing Aβ generation, anti-apoptosis, antioxidant, and promoting nerve repair. Therefore, in the development of medicaments for AD, natural products have attracted widespread attention from domestic and foreign researchers due to their biological activity and structural diversity. In 2019, China's drug administration organization conditionally approved the marketing of sodium oligomannate for the treatment of mild to moderate AD. The success of sodium oligomannate has inspired Chinese scholars to search for medicaments for the prevention or treatment of neurodegenerative diseases from natural products.

Chlorogenic acid (CGA), also known as caffeotannic acid, is a phenolic acid condensated by caffeic acid (CA) and quinic acid (QA), with a chemical name of 3-O-caffeoylquinic acid (CGA). Chlorogenic acid is a phenylpropanoid synthesized by intermediate products of the pentose phosphate pathway during aerobic respiration in plants. Chlorogenic acid has been developed and applied in a few of fields such as food, health products, cosmetics, and pharmaceuticals. CGA is widely present in many common vegetables and fruits, and has various biological activities, such as cardiovascular protection, antioxidant, anti-UV and anti-radiation, anti-mutagenic and anti-cancer, antibacterial, antiviral, lipid-lowering and hypoglycemic, immune regulatory properties, etc. It has already been widely used in fields such as medicine, chemical industry, and food.

Previously, it has already been shown that CGA plays antigenic, antioxidant and other activities, protects nerve cells, reduces the risk of cognitive decline and neuropathy, and thus mitigates clinical features of AD by improving memory disorders and cognitive dysfunction, inhibiting β-amyloid induced neurotoxicity, and alleviating mitochondrial damage and apoptosis, (see, for example, Yin-Le Yao et al., “Research progress on chlorogenic acid improving Alzheimer's disease”, Herald of Medicine, Volume 36, Issue 11, November 2017, pp. 1287-1290). However, there is relatively little research on the specific effects of CGA in clinical practice, and previously, there have been no studies on which stage of Alzheimer's disease chlorogenic acid effectively acts, such as mild, moderate, and severe AD.

The team of Sichuan Jiuzhang Biological Science and Biotechnology Co., Ltd., the present inventors, has been conducting systematic research and development on CGA since 2000. In August 2013, we obtained clinical approvals for CGA raw materials and injections (approval certificate numbers: 2013L01855, 2013L01856). In December 2016, the “Phase I/II clinical study of CGA for injection, the first Class 1 new drug” was included in the 13th Five Year Major Scientific and Technological Special Project for “Significant New Drugs Development” by the National Health and Family Planning Commission and the Ministry of Science and Technology, and is also a major science and technology support project in Sichuan Province.

The inventor has long been dedicated to research on the use of CGA in the treatment of AD. In the in-depth study of the pharmacological effects of CGA, the inventor unexpectedly discovered a method that can significantly improve the efficacy of CGA in the treatment of early AD by extensive screening, and developed a safe and efficient pharmaceutical composition containing CGA that is particularly suitable for the treatment of early AD.

### SUMMARY OF THE INVENTION

In order to solve the above-mentioned technical problems, the present invention significantly enhances the therapeutic effect of CGA on early AD by combining the main active ingredient CGA with the auxiliary ingredient 3-coumaroylquinic acid in a specific dosage ratio, and develops a safe and efficient pharmaceutical composition containing CGA that is particularly suitable for the treatment of early AD.

Specifically, the present invention has been implemented by the following technical solutions:

In a first aspect, the present invention provides a pharmaceutical composition for treating early AD, which comprises CGA and 3-coumaroylquinic acid, with a weight ratio of 100:(0.01-0.5), wherein CGA is the main active ingredient for treating early AD, while 3-coumaroylquinic acid is an auxiliary ingredient used to enhance the efficacy of CGA in the treatment of early AD.

Alternatively, in the above pharmaceutical composition, the weight ratio of CGA to 3-coumaroylquinic acid is 100:0.05.

Alternatively, in the above pharmaceutical composition, the pharmaceutical composition is selected from one or more oral or non-oral preparations.

Alternatively, in the above pharmaceutical composition, the oral preparation is one or more of capsules, tablets, oral liquids, or granules.

Preferably, the oral preparation is orally disintegrating tablets (ODTs) that are convenient for the elderly to take.

Alternatively, in the above pharmaceutical composition, the non-oral preparation is one or more of injections, creams, patches, ointments, suppositories or sprays.

Alternatively, in the above pharmaceutical composition, the administration route of the injection is one or more of subcutaneous administration, intramuscular administration, or intravenous administration.

Alternatively, in the above pharmaceutical composition, the pharmaceutical composition further comprises other active ingredients selected from one or more of donepezil, galantamine, rivastigmine, memantine, prednisone, rofecoxib, nimesulide, diclofenac, rhodanine, Ginkgo biloba leaf extract, ginsenoside, huperzine A, stilbene glycoside, levodopa, carbidopa, benserazide, trihexyphenidyl, benzotropine, procycline, prdenamhe, butylphthalide, dipyridamole, low molecular weight dextran, heparin, urinary kallidinogenase, citicoline, butylphthalide, edaravone, nimodipine, or aspirin.

In a second aspect, the present invention provides the use of the pharmaceutical composition mentioned in the above first aspect in the manufacturer of medicaments for the treatment of early AD.

Alternatively, in the above use, the medicament improves the learning and memory abilities of patients with early AD, delays the progression of AD, and raises the life quality of patients.

Compared to the prior art, the present invention has the following beneficial effects: in the present invention, the main active ingredient CGA is combined with the auxiliary ingredient 3-coumaroylquinic acid in a specific dosage ratio, which can effectively enhance the therapeutic effect of CGA on early AD, thereby greatly reducing the dosage of CGA, but can achieve better therapeutic effects than treatment of early AD with CGA alone.

### EXAMPLES

In the in-depth study on the pharmacological effects of CGA, the inventors unexpectedly discovered a method that could significantly improve the efficacy of CGA in the treatment of early AD by extensive screening, and developed a pharmaceutical composition containing CGA that was particularly suitable for the treatment of early AD. On this basis, the present invention had been completed.

In order to better understand the present invention, further explanations of some terms involved in the present invention is provided below.

“Early Alzheimer's disease” or “mild Alzheimer's disease” can be diagnosed and classified as “possible Alzheimer's disease” according to the evaluation criteria in the “Diagnosis and Treatment Guidelines for Alzheimer's Disease (2020 Edition)” issued by the National Health Commission, PRC.

As used herein, “auxiliary ingredients” usually refer to substances that do not have or have almost no target pharmacological activity, but can enhance the target pharmacological activity of the main active ingredient. The main pharmacological activity of the present invention is to treat early AD.

As used herein, “3-coumaroylquinic acid” is a possible trace impurity present in the extraction process of CGA. The raw materials of CGA extracted using the extraction method developed previously by our company contain 3-coumaroylquinic acid, 5-caffeoylquinic acid (neochlorogenic acid), 4-vinylcatechol (decarboxylated product of caffeic acid), 4-caffeoylquinic acid (cryptochlorogenic acid), and methylation derivatives of CGA, with a total impurity content of <1.5% (see CN105085265B). Previously, our company has creatively attempted to combine “chlorogenic acid” with “coumaroylquinic acid” to play synergistic anti-tumor effects, specifically involving the manufacturer of medicaments for the treatment of multidrug-resistant tumors (CN108159028B), the treatment of sarcomas (CN108653263B), the treatment of squamous cell carcinoma (CN108685892B), and the treatment of renal cancer (CN108498497B), all of which have been successful. However, there are currently no reports of combining CGA with 3-coumaroylquinic acid for the treatment of early AD.

As used herein, the term “pharmaceutical composition” refers to a composition that can be applied to a mammalian host, for example, by oral, local, parenteral, inhalation spray or rectum, in the form of a unit dosage form comprising commonly used non-toxic carriers, diluents, adjuvants, mediators, etc. The term “parenteral” used herein includes subcutaneous injection, intravenous injection, intramuscular injection, or infusion techniques.

A pharmaceutical composition comprising compounds of the present invention may be in a form suitable for oral administration, such as tablets, pills, troches, dispersible powders or granules, emulsions, hard or soft capsules, syrups or elixirs.

The composition intended for oral administration can be manufactured according to any known method, and may include one or more agents selected from sweeteners, flavoring agents, colorants, and preservatives, to provide a pharmaceutically pleasing and palatable formulation. Tablets may contain a mixture of active ingredients and non-toxic pharmaceutically acceptable excipients suitable for preparation of tablets. These excipients can be, for example, inert diluents such as calcium carbonate, sodium carbonate, lactose, calcium phosphate, or sodium phosphate; granulating agents and disintegrants, such as corn starch or alginic acid; adhesives, such as starch, gelatin, or arabic gum; and lubricants such as magnesium stearate, stearic acid, or talc powder. Tablets can be uncoated or coated with well-known techniques to delay disintegration and absorption in the gastrointestinal tract, and thus provide sustained action for a longer period of time. For example, controlled or sustained materials can be used, such as glyceryl monostearate or glyceryl distearate. The tablets can also be osmotic pump controlled release tablets.

Formulations for oral administration can also be made into hard capsules, wherein the active ingredients are mixed with inert solid diluents such as calcium carbonate, calcium phosphate, or kaolin; or made into soft capsules, wherein the active ingredients are mixed with water or oil media such as peanut oil, liquid paraffin, or olive oil. Syrups and elixirs can be made by mixing with sweeteners such as glycerol, propylene glycol, sorbitol, or sucrose. Such preparations can also include alleviants, preservatives, flavoring agents, and colorants.

Sterile injectable preparations can also be sterile injectable solutions or suspensions in non-toxic and parenterally acceptable diluents or solvents, such as solutions in 1,3-butanediol. Among acceptable media and solvents, Ringer's solution and isotonic NaCl solution can be used.

The composition can also be in the form of a suppository for rectal administration of the compound according to the present invention. These compositions can be prepared by mixing drugs with suitable non-irritating excipients, which are solid at room temperature and liquid at rectal temperature, and can thus melt in the rectum to release the drug. For example, such materials include cocoa butter and polyethylene glycol.

For local administration, focus is placed on creams, ointments, gel, solutions, lotions, absorbent dressings, aerosols, etc., comprising the compounds of the present invention. These topical formulations can contain commonly used suitable additives, such as preservatives, solvents for assisting drug penetration, and softeners in ointments and creams. These formulations can also include commonly used carriers which are compatible, such as cream or ointment matrices, as well as ethanol or oleyl alcohol used in lotions. Topical preparations should include mouth rinse and mouthwash.

In order to facilitate the delivery of the compounds according to the present invention from a pressurized cartridge or spray in the form of aerosols for inhalation administration, suitable propellants such as dichlorodifluoromethane, trichlorofluoromethane, dichlorotetrafluoroethane, tetrafluoroethane, heptafluoropropane, carbon dioxide or other suitable gases are used.

In the case of pressurized aerosols, the dosage unit can be determined by installing valves to deliver the measured amount. Gelatin capsules and cartridges, such as those used for inhalers or insufflators, can be prepared with powder mixtures containing compounds of the present invention and suitable powder matrices such as lactose or starch.

The various dosage forms mentioned above can be manufactured according to conventional processes in the field of pharmaceutical preparations.

The present invention will be described by reference to the following example, which is merely illustrative and are not to be construed as a limitation of the scope of the present invention.

If specific technology or conditions are not specified in the example, the technology or conditions described in the literature in this field, or the manufacturer's instructions should be followed. The reagents or instruments used without the manufacturer's indication are conventional products that can be commercially available.

Unless otherwise specified, the experimental methods in the following example are all conventional methods. The experimental materials used in the following example, unless otherwise specified, are all those commercially available.

Unless otherwise specified, the percentages and parts referred to in the present invention are percentages by weight and parts by weight.

### Example: Pharmacodynamic Study on the Pharmaceutical Composition of the Present Invention for the Treatment of Early AD

In the clinic, many clinical epidemiological studies on AD have shown that cerebral ischemia, especially chronic cerebral hypoperfusion commonly found in the elderly, is closely related to the occurrence and development of AD. The cerebral arterial circle was taken out immediately after the death of the patients with AD, and the stenosis degree of the vascular lumen was detected. The results showed that the levels of atherosclerosis were significantly higher than the age-matched control group, and the vascular stenosis index was highly correlated with the characteristic neuropathological changes of AD. Research on cerebral blood flow and metabolism in the early stages of AD suggests that the decrease in cerebral blood flow and cerebral hypometabolism caused by cerebral ischemia occurs earlier than the neuropathological changes and dementia manifestations of AD. In the case of chronic cerebral hypoperfusion, the deposition of Aβ in the brain indicates that chronic cerebral hypoperfusion is an important risk factor for initiating the onset of AD. The systematic study on the impact of chronic cerebral hypoperfusion on the onset of AD has become an important target for the prevention and treatment of AD

### 1. Experimental Objective

In this experiment, the occurrence of mild AD in humans after chronic cerebral hypoperfusion was simulated by performing bilateral carotid artery ligation in rats, and the intervention of the pharmaceutical composition according to the present invention on the learning and memory ability of these rats was observed, in order to investigate the therapeutic effect of the pharmaceutical composition according to the present invention on early AD.

### 2. Experimental Materials

### 2.1. Experimental Animals: 6-8 Week Old, SPF Grade, Male Sprague Dawley (SD) Healthy Rats, Weighing 280-300 g.

### 2.2. Test Drug:

CGA and 3-coumaroylquinic acid (extracted and purified from Eucommia ulmoides leaves, with a purity of over 99.5%) were self-made by Sichuan Jiuzhang Biological Science and Biotechnology Co., Ltd. . . . Sodium oligomannate capsules (150 mg, national medicine permission number H20190031) were purchased from Shanghai Greenvalley Pharmaceutical Co. Ltd.

### 3. Experimental Methods

### 3.1. Establishment of a Chronic Cerebral Hypoperfusion Model

A chronic cerebral hypoperfusion model was established by bilateral carotid artery ligation. Rats were acclimated to the environment for 1 week, and then fasted for 12 h and water-deprivated for 4 h before surgery. The rats were anesthetized with 0.4% pentobarbital sodium (0.5 mL/100 g i.p.), and fixed in a supine position on the operating table. Hair was removed from the skin in front of the neck. The naked skin was disinfected with iodine, and then the rats were incised in the middle of the neck. The subcutaneous superficial fascia was bluntly separated, and then the sternocleidomastoideole and sternohyoid muscle were carefully separated along both sides of the trachea, with arterial sheath visible; the bilateral common carotid arteries were separated and exposed with a glass needle, to bury the thread, and then double ligated with silk thread. The blood vessels between the two ligation points were cut off. The incision was sutured layer by layer and disinfected, followed by waiting for awakening of rats. During the operation, incandescent lamp irradiation was used to maintain the anal temperature of rats at 36.5° C.-37.5° C. After surgery, each rat was placed in a single cage to prevent suffocation.

### 3.2. Grouping and Administration of Experimental Animals

10 healthy SD rats with suitable body weight (within the required range) were randomly selected as the normal control group. 80 successfully modeled rats were randomly divided into 8 groups, with 10 rats in each group. They were named as follows: model control group (n=10), CGA treatment group (n=10), 3-coumaroylquinic acid treatment group (n=10), CGA+3-coumaroylquinic acid (100:0.01) treatment group (n=10), CGA+3-coumaroylquinic acid (100:0.05) treatment group (n=10), CGA+3-coumaroylquinic acid (100:0.5) treatment group (n=10), CGA+3-coumaroylquinic acid (100:1) treatment group (n=10), sodium oligomannate treatment group (n=10), and thus, there are 9 experimental groups, along with the normal control group (n=10), for a total of 90 rats.

In this experiment, except for sodium oligomannate treatment group, which was administered by gavage, all other treatment groups were administered by intraperitoneal injection to each group of rats. The blank group and model group were given the solvent physiological saline by intraperitoneal injection. Rats in each treatment group were administrated one week after modeling surgery. Then, after 20 consecutive days of administration, the experiment was carried out. The administration continued during the experiment. At the same time, both the blank group and the model group were given an equal amount of physiological saline. The composition and dosage for each treatment group were as follows:
(1) CGA treatment group: CGA, 60 mg/kg/d, administered by intraperitoneal injection using physiological saline as the solvent.
(2) 3-coumaroylquinic acid treatment group: 3-coumaroylquinic acid, 0.03 mg/kg/d, administered by intraperitoneal injection using physiological saline as the solvent.
(3) CGA+3-coumaroylquinic acid (100:0.01) treatment group: CGA (60 mg/kg/d)+3-coumaroylquinic acid (0.006 mg/kg/d), administered by intraperitoneal injection using physiological saline as the solvent.
(4) CGA+3-coumaroylquinic acid (100:0.05) treatment group: CGA (60 mg/kg/d)+3-coumaroylquinic acid (0.03 mg/kg/d), administered by intraperitoneal injection using physiological saline as the solvent.
(5) CGA+3-coumaroylquinic acid (100:0.5) treatment group: CGA (60 mg/kg/d)+3-coumaroylquinic acid (0.3 mg/kg/d), administered by intraperitoneal injection using physiological saline as the solvent.
(6) CGA+3-coumaroylquinic acid (100:1) treatment group: CGA (60 mg/kg/d)+3-coumaroylquinic acid (0.6 mg/kg/d), administered by intraperitoneal injection using physiological saline as the solvent.
(7) Sodium oligomannate treatment group: sodium oligomannate, 100 mg/kg/d, administered by gavage using physiological saline as the solvent.

### 3.3. Observation Indexes

Morris water maze behavior test was performed after administration. The water maze consisted of a circular pool, an automatic camera, and a computer analysis system. The camera above the pool synchronously recorded the movement trajectory of the rats. Before the test, water was poured into the pool, so as to have a water depth of 30 cm. The water surface was kept at 1 cm above the platform surface. Milk powder was added to make water become milky white, so that rats could not see the platform. The water temperature was kept between 22-25° C. One day before the experiment, the head of each rat was dyed black, and each animal was trained twice on the first day to learn and remember. The entry position was in the opposite-quadrant and adjacent-quadrants of the platform, and each rat was allowed to enter water with the animal's head directing towards the pool wall at ½ arc of the quadrant edge. If it could not find the platform within 120 seconds, the rat was led to the platform and left to learn and remember for 30 seconds. During the training period, the environment remained quiet, and the reference object was not changed.

### (1) Orientation Navigation Experiment

The experiment was carried out for a total of 5 days. The data collection and image analysis were all completed by the image automatic monitoring and processing system. After the experiment, the swimming time and distance of each animal arriving at the platform, the initial angle, the trajectory of searching for the platform, and the escape latency were recorded. The time limit for the escape latency was set to be 120 seconds.

### (2) Space Exploration Experiment

After completion of the orientation navigation experiment, the underwater platform was removed, and rats were placed in water with their heads facing the pool wall at the same entry point. The rats were allowed to search for the platform in their memory without the platform, and their staying time in the original platform quadrant was recorded.

### 3.4. Statistical Methods

The data were analyzed using SPSS 18.0 statistical software, and the measurement data were expressed as mean±standard deviation (SD). One-way analysis of Variance was used for groups comparison. Two-way repeated measures ANOVA was used for analysis of the data obtained from the orientation navigation experiment in water maze, and LSD test was used for the pairwise multiple comparison. P<0.05 indicated a statistically significant difference.

### 4. Experimental Results

### 4.1. The Effect of the Pharmaceutical Composition According to the Present Invention on the Orientation Navigation of Rats in Morris Water Maze Experiment

As shown in Tables 1 and 2, the statistical analysis results of Two-way repeated measures ANOVA and the pairwise multiple comparison showed that compared with the blank control group, the escape latency of the model group rats was significantly prolonged (p<0.01), and the searching distance was significantly increased (p<0.01), indicating that the chronic cerebral hypoperfusion model could simulate the learning and memory deficits in early AD.

After analyzing the experimental results of each treatment group, it was found that compared with the model group, the escape latency and searching distance of rats in the CGA treatment group and 3-coumaroylquinic acid treatment group showed almost no improvement, indicating that CGA alone and 3-coumaroylquinic acid alone had no notable improvement effect on the learning and memory ability of experimental rats. However, the effect of CGA alone was slightly better than that of 3-coumaroylquinic acid alone.

After analyzing the experimental results of the combination groups of CGA and 3-coumaroylquinic acid at different weight ratios, it was found that, different from the expected results, that is, the higher dosage of 3-coumaroylquinic acid did not mean that the enhancement effect on CGA was stronger. When the weight ratio of CGA to 3-coumaroylquinic acid in the composition was 100:0.05, the synergistic effect of 3-coumaroylquinic acid on CGA was the strongest. During the experiment, with the passage of time, the escape latency and searching distance of rats were significantly shortened compared to the model group (p<0.01), and its effect was comparable to the positive control drug sodium oligomannate (p<0.01), and even better than sodium oligomannate at certain time points. In addition, when the weight ratio of CGA to 3-coumaroylquinic acid in the composition was 100:0.01 or 100:0.5, although the synergistic effect of 3-coumaroylquinic acid on CGA was not as significant as the weight ratio of 100:0.05, during the experiment, the escape latency and searching distance of rats were significantly shortened over time compared to the model group (p<0.05). However, surprisingly, when the dosage of 3-coumaroylquinic acid in the composition was further increased, and the weight ratio of CGA and 3-coumaroylquinic acid was 100:1, 3-coumaroylquinic acid did not show a significant synergistic effect on CGA.

TABLE 1
Effect of the pharmaceutical composition according the present invention
on the average escape latency in Morris water maze experiment (s).

| Groups | Day 1 | Day 2 | Day 3 | Day 4 | Day 5 |
|---|---|---|---|---|---|
| Blank group | 68.5 ± 12.4 | 62.3 ± 13.5 | 45.6 ± 12.2 | 21.4 ± 12.1 | 14.7 ± 5.8 |
| Model group | 99.7 ± 18.1^{#} | 95.3 ± 14.6^{##} | 85.4 ± 13.9^{##} | 77.1 ± 15.2^{##} | 54.6 ± 12.9^{##} |
| CGA group | 95.4 ± 15.1 | 89.2 ± 17.5 | 75.6 ± 12.6 | 70.7 ± 14.5 | 46.3 ± 10.6 |
| 3-coumaroylquinic acid | 98.1 ± 16.8 | 94.2 ± 15.3 | 81.5 ± 13.7 | 75.4 ± 16.3 | 51.8 ± 14.2 |
| group | | | | | |
| CGA + | 93.4 ± 11.6 | 80.3 ± 11.8 | 63.7 ± 10.1* | 38.9 ± 10.4* | 27.6 ± 7.3* |
| 3-coumaroylquinic acid | | | | | |
| group (100:0.01) | | | | | |
| CGA + | 90.1 ± 12.3 | 70.4 ± 15.1* | 47.4 ± 11.3** | 25.3 ± 9.9** | 17.2 ± 6.8** |
| 3-coumaroylquinic acid | | | | | |
| group (100:0.05) | | | | | |
| CGA + | 94.8 ± 10.5 | 79.2 ± 12.1 | 60.4 ± 9.9* | 35.2 ± 8.1* | 26.5 ± 6.9* |
| 3-coumaroylquinic acid | | | | | |
| group (100:0.5) | | | | | |
| CGA + | 96.2 ± 17.4 | 85.5 ± 15.9 | 71.6 ± 14.8 | 65.2 ± 13.4 | 40.1 ± 12.5 |
| 3-coumaroylquinic acid | | | | | |
| group (100:1) | | | | | |
| Sodium oligomannate | 88.3 ± 10.7 | 68.4 ± 12.9** | 49.2 ± 13.2** | 26.1 ± 10.1** | 19.3 ± 7.2** |
| group | | | | | |

Note:
^{#}p < 0.05,
^{##}p < 0.01, comparing the model group and the blank group;
*P < 0.05,
**p < 0.01, comparing the treatment group and the model group.

TABLE 2
Effect of the pharmaceutical composition according the present invention
on the searching distance in Morris water maze experiment (cm).

| Groups | Day 1 | Day 2 | Day 3 | Day 4 | Day 5 |
|---|---|---|---|---|---|
| Blank group | 852.6 ± 105.3 | 574.7 ± 114.6 | 283.6 ± 109.7 | 198.4 ± 71.2 | 121.2 ± 50.4 |
| Model group | 1205.1 ± 134.5^{#} | 966.2 ± 122.9^{##} | 904.5 ± 113.6^{##} | 580.2 ± 68.1^{##} | 420.1 ± 48.7^{##} |
| CGA group | 1125.6 ± 108.4 | 944.3 ± 102.5 | 844.8 ± 99.1 | 560.9 ± 64.2 | 395.2 ± 42.7 |
| 3-coumaroylquinic acid group | 1127.3 ± 117.2 | 954.5 ± 119.3 | 892.6 ± 106.2 | 577.1 ± 72.5 | 407.1 ± 44.3 |
| CGA + 3-coumaroylquinic acid | 1090.8 ± 107.2 | 793 ± 96.5 | 682.4 ± 104.1* | 396.1 ± 68.7* | 262.5 ± 50.6* |
| group (100:0.01) | | | | | |
| CGA + 3-coumaroylquinic acid | 1033.9 ± 102.6 | 695 ± 111.6* | 511.2 ± 106.7** | 252.6 ± 69.8** | 150.5 ± 40.4** |
| group (100:0.05) | | | | | |
| CGA + 3-coumaroylquinic acid | 1087.2 ± 101.4 | 780 ± 104.2 | 649.3 ± 109.8* | 387.4 ± 66.4* | 239.6 ± 46.1* |
| group (100:0.5) | | | | | |
| CGA + 3-coumaroylquinic acid | 1105.1 ± 112.3 | 900.7 ± 99.4 | 792.5 ± 92.7 | 520.1 ± 69.7 | 343.1 ± 48.9 |
| group (100:1) | | | | | |
| Sodium oligomannate group | 998.4 ± 108.7 | 684 ± 106.4* | 520.5 ± 91.3** | 287.3 ± 52.9** | 144.3 ± 37.1** |

Note:
^{#}p < 0.05,
^{##}p < 0.01, comparing the model group and the blank group;
*P < 0.05,
**p < 0.01, comparing the treatment group and the model group.

### 4.2. The Effect of the Pharmaceutical Composition According to the Present Invention on the Spatial Seeking Ability of Rats in Morris Water Maze Experiment

As shown in Table 3 below, the statistical analysis results of two-way repeated measures ANOVA and the pairwise multiple comparison showed that compared with the rats in the blank control group, the rats in the model group had significantly shorter searching time in the quadrant where the original platform was located (p<0.01), indicating that the chronic cerebral hypoperfusion model could simulate the decline of learning and memory ability in early AD.

After analyzing the experimental results of each treatment group, it was found that compared with the model group, the searching time of rats in the CGA treatment group and 3-coumaroylquinic acid treatment group in the quadrant of the original platform showed almost no extension, indicating that CGA alone and 3-coumaroylquinic acid alone had no notable improvement effect on the learning and memory ability of experimental rats. However, the effect of CGA alone was slightly better than that of 3-coumaroylquinic acid alone.

After analyzing the experimental results of the combination groups of CGA and 3-coumaroylquinic acid at different weight ratios, it was found that, different from the expected results, that is, the higher the dosage of 3-coumaroylquinic acid, the stronger the enhancement effect on CGA. When the weight ratio of CGA and 3-coumaroylquinic acid in the composition was 100:0.05, the synergistic effect of 3-coumaroylquinic acid on CGA was the strongest. During the experiment, the searching time of rats in the quadrant of the original platform was significantly longer than that of the model group (p<0.01), and its effect was comparable to that of the positive control drug, sodium oligomannate (p<0.01). In addition, when the weight ratio of CGA to 3-coumaroylquinic acid in the composition was 100:0.01 or 100:0.5, although the synergistic effect of 3-coumaroylquinic acid on CGA was not as good as the weight ratio of 100:0.05, during the experiment, the searching time of rats in the quadrant where the original platform was located was significantly longer than that of the model group (p<0.05). However, surprisingly, when the dosage of 3-coumaroylquinic acid in the composition was further increased, and the weight ratio of CGA and 3-coumaroylquinic acid was 100:1, 3-coumaroylquinic acid did not show a significant synergistic effect on CGA.

TABLE 3
Effect of the pharmaceutical composition according
to the present invention on the spatial seeking
ability in Morris water maze experiment.

| | Time spent in the original |
|---|---|
| Groups | platform quadrant (s) |
| Blank group | 60.2 ± 10.1 |
| Model group | 29.4 ± 8.9^{##} |
| CGA group | 32.6 ± 8.5 |
| 3-coumaroylquinic acid group | 30.2 ± 7.8 |
| CGA + 3-coumaroylquinic acid group | 40.7 ± 8.6* |
| (100:0.01) | |
| CGA + 3-coumaroylquinic acid group | 46.3 ± 9.5* |
| (100:0.05) | |
| CGA + 3-coumaroylquinic acid group | 41.5 ± 9.1* |
| (100:0.5) | |
| CGA + 3-coumaroylquinic acid group | 34.3 ± 9.2 |
| (100:1) | |
| Sodium oligomannate group | 48.5 ± 9.4** |

Note:
^{#}p < 0.05,
^{##}p < 0.01, comparing the model group and the blank group;
*P < 0.05,
**p < 0.01, comparing the treatment group and the model group.

### 5. Experimental Conclusion

The above experimental results indicated that the pharmaceutical composition of the present invention comprising CGA and 3-coumaroylquinic acid at a specific weight ratio could significantly improve the spatial learning and memory ability of chronic cerebral hypoperfusion model rats.

Therefore, CGA was used as the main active ingredient in the pharmaceutical composition according to the present invention, while 3-coumaroylquinic acid was used as an auxiliary ingredient, which could effectively enhance the therapeutic effect of CGA on early AD. Therefore, the research and development of the pharmaceutical composition according to the present invention was of great significance for early intervention in AD, delaying the progression of AD, and improving the life quality of patients.

Obviously, based on the teachings of the present invention, those skilled in the art may make various modifications and variations, without departing from the basic spirit and scope of the present disclosure. All such modifications and variations coming within the scope of the appended claims and their equivalents are intended to be included therein.

1. A pharmaceutical composition for treating early Alzheimer's disease, characterized in that the pharmaceutical composition comprises chlorogenic acid and 3-coumaroylquinic acid, with a weight ratio of 100:(0.01-0.5), wherein chlorogenic acid is the main active ingredient for treating early Alzheimer's disease, while 3-coumaroylquinic acid is an auxiliary ingredient used to enhance the efficacy of chlorogenic acid in the treatment of early Alzheimer's disease.

2. The pharmaceutical composition according to claim 1, characterized in that the weight ratio of chlorogenic acid to 3-coumaroylquinic acid is 100:0.05.

3. The pharmaceutical composition according to claim 1, characterized in that the pharmaceutical composition is selected from one or more oral or non-oral preparations.

4. The pharmaceutical composition according to claim 3, characterized in that the oral preparation is one or more of capsules, tablets, oral liquids, or granules, and preferably, the oral preparation is orally disintegrating tablets (ODTs).

5. The pharmaceutical composition according to claim 3, characterized in that the non-oral preparation is one or more of injections, creams, patches, ointments, suppositories or sprays.

6. The pharmaceutical composition according to claim 5, characterized in that the administration route of the injection is one or more of subcutaneous administration, intramuscular administration, or intravenous administration.

7. A pharmaceutical composition according to claim 1, characterized in that: the pharmaceutical composition further comprises other active ingredients selected from one or more of donepezil, galantamine, rivastigmine, memantine, prednisone, rofecoxib, nimesulide, diclofenac, rhodanine, Ginkgo biloba leaf extract, ginsenoside, huperzine A, stilbene glycoside, levodopa, carbidopa, benserazide, trihexyphenidyl, benzotropine, procycline, prdenamhe, butylphthalide, dipyridamole, low molecular weight dextran, heparin, urinary kallidinogenase, citicoline, butylphthalide, edaravone, nimodipine, or aspirin.

8. The pharmaceutical composition according claim 1 for use in the manufacturer of medicaments for the treatment of early Alzheimer's disease.

9. The use according to claim 8, characterized in that the medicament improves the learning and memory abilities of patients with early Alzheimer's disease, delays the progression of Alzheimer's disease, and raises the life quality of patients.
